(19) United States
(12) Patent Application Publication
(10) Pub. No.: US 2023/0100584 A1
(43) Pub. Date: Mar. 30, 2023

(54) PREDICTING FORMULATION PROPERTIES

(71) Applicant: Bayer Aktiengesellschaft, Leverkusen (DE)

(72) Inventors: Holger DIEDAM, Köln (DE); Johanna ANLAHR, Dortmund (DE); Heike SKÖTSCH, Berlin (DE); Matthias ERBER, Düsseldorf (DE)

(73) Assignee: Bayer Aktiengesellschaft, Leverkusen (DE)

(21) Appl. No.: 17/800,449

(22) PCT Filed: Feb. 18, 2021
(86) PCT No.: PCT/EP2021/054006

§ 371 Date: Aug. 17, 2022

(30) Foreign Application Priority Data
Feb. 19, 2020 (EP) 20158149.3

(51) Int. Cl. G16C 20/30; G16C 20/70; G06N 3/09

(52) U.S. Cl. CPC G16C 20/30; G16C 20/70; G06N 3/09

(57) ABSTRACT

The invention relates to the development of formulations, preferably for biologically active substances. The aim of the invention is to provide a method, a computer system, and a computer program product for predicting at least one property of at least one formulation using a prediction model which has been trained to predict formulation properties by means of a monitored learning process using reference data.

### CROSS REFERENCE TO RELATED APPLICATIONS

This application is a national stage application under 35 U.S.C. § 371 of International Application No. PCT/EP2021/054006, filed internationally on Feb. 18, 2021, which claims priority to and benefit of EP Patent Application No. 20158149.3, filed Feb. 19, 2020, the disclosures of which are hereby incorporated herein by reference in their entirety.

### FIELD

The present invention relates to the development of formulations, preferably for biologically active substances. In particular, the present invention relates to a method, a computer system, and a computer program product for predicting at least one property of at least one formulation using a prediction model that has been trained to predict formulation properties by means of supervised learning using reference data.

### BACKGROUND

A formulation is a mixture of different substances. A formulation is produced from defined amounts of the substances according to a formula. A formulation usually serves to bring one or more constituents (substances) of the formulation into a form that meets a defined purpose. In the case of medicaments, plant protection agents, and pest control agents, an active ingredient is usually brought into a form using formulation auxiliaries that improves the biological effect of the active ingredient in a target organism compared to the pure active ingredient and/or makes the finished product usable.

When developing a formulation, different formulations are typically generated, and their properties may be tested and compared with one another in order to find a formulation suitable for a defined purpose. The aim is usually to find the “optimal” formulation for the intended application, with the optimization often taking place in relation to various target parameters (such as cost, environmental compatibility, bioavailability, handling, etc.).

### SUMMARY

In order to reduce the experimental effort involved in developing a novel formulation, it would be desirable if decision-making aids were available for the selection of possible formulations.

The present disclosure attends to this aim. The subject matter disclosed herein provides means that may be used in the development of novel formulations in order to reduce experimental effort and/or to be able to carry out targeted investigations. Preferred embodiments of the invention are may be found in the dependent claims, in the present description and in the figures.

In some embodiments, a computer system is provided. The computer system may comprise:
an input unit,
a control and computation unit, and
an output unit,
   wherein the control and computation unit is configured
to prompt the input unit to receive a unique identifier of a substance,
to determine substance properties based on the unique identifier,
to generate a feature vector for the substance based on the substance properties,
using the feature vector to calculate at least one formulation property of at least one formulation using a prediction model, the prediction model having been trained in a supervised learning process to calculate formulation properties for reference formulations using reference data from reference substances, and
to prompt the output unit to output the at least one formulation property.

In some embodiments, a method is provided. The method may comprise:
receiving a unique identifier of a substance,
receiving and/or determining substance properties of the substance,
generating a feature vector for the substance based on the substance properties,
supplying the feature vector to a prediction model, the prediction model having been trained in a supervised learning process to determine formulation properties for reference formulations using reference data from reference substances,
receiving at least one formulation property for at least one formulation comprising the substance as an output from the prediction model, and
outputting the at least one formulation property.

In some embodiments, a computer program product is provided. The computer program product may comprise a data carrier on which there is stored a computer program that can be loaded into the main memory of a computer system, where it prompts the computer system to execute the following steps:
receiving a unique identifier of a substance,
receiving and/or determining substance properties of the substance,
generating a feature vector for the substance based on the substance properties,
determining at least one formulation property for at least one formulation of the substance using the feature vector by means of a prediction model, the prediction model having been trained in a supervised learning process to determine formulation properties for reference formulations using reference data from reference substances, and
outputting the at least one formulation property.

### BRIEF DESCRIPTION OF THE FIGURES

FIG. 1 shows an exemplary embodiment of the computer system in accordance with some embodiments.

FIG. 2 shows an exemplary embodiment of the method which is carried out by the computer system in accordance with some embodiments.

FIG. 3 shows an exemplary graphical user interface that can be provided by the computer system in accordance with some embodiments.

FIG. 4 shows an exemplary result of the prediction of formulation properties for a substance in the form of a graphical representation in accordance with some embodiments.

### DETAILED DESCRIPTION

The invention will be more particularly elucidated below without distinguishing between the subjects of the invention (computer system, method, computer program product). The elucidations that follow shall instead apply analogously to all subjects of the invention, regardless of the context in which they are made (computer system, method, computer program product).

If steps are stated in an order in the present description or in the claims, this does not necessarily mean that the invention is restricted to the stated order. On the contrary, it is possible for the steps also to be able to be executed in a different sequence or in parallel to one another, unless one step builds upon another step, which by definition means that the step building upon the other is executed subsequently (but this will be clear in the individual case). The orders stated are thus preferred embodiments of the invention.

The starting point of the present invention is a substance for which a suitable formulation is to be determined.

In some embodiments, a substance in the context of the present invention may be a chemical compound in the form of a pure substance which has a defined chemical structure. The chemical structure may reflect the structure at the molecular or ionic level. In some embodiments, the substance may be a solid substance under standard conditions (temperature: 273.15K, pressure: 1 bar). In some embodiments, the substance may be in liquid form under standard conditions. In some embodiments, the substance may be in solid form under standard conditions and at room temperature (25° C.).

In some embodiments, the substance may be an organic compound. An organic compound is a chemical compound comprising carbon-hydrogen bonds (C—H bonds). In some embodiments, preference may be given to an organic compound, the molecules of which are formed solely from the following elements: carbon (C), hydrogen (H), oxygen (O), nitrogen (N), sulfur (S), fluorine (F), chlorine (Cl), bromine (Br), iodine (I) and/or phosphorus (P).

In some embodiments, the substance may be a biologically active substance. Biologically active substances, also referred to as active ingredients, are substances that have a specific effect or cause a specific reaction in a biological organism. Examples of such biologically active substances are medicaments (pharmaceutically active substances), plant protection agents (e.g. pesticides, herbicides, fungicides), or (other) pest control agents (biocides such as insecticides, bactericides, nematicides and the like).

In some embodiments, the substance may be a biologically active organic compound.

A formulation is a mixture of chemical compounds that comprises further (auxiliary) substances in addition to the substance. In creating a formulation, the substance may be put into a form that is particularly appropriate for its intended application (purpose). In the case of a formulation, the intended application (purpose) is therefore decisive for the properties to be optimized.

In some embodiments, the formulation may be a pharmaceutically active ingredient formulation which aims to increase the solubility in a defined medium and/or the bioavailability of the active ingredient and/or adjust the release rate in a defined manner.

In some embodiments, the formulation may be a phytomedical preparation of a pesticide with adjuvants (formulation adjuvants) in order to enable application and/or good distribution to be as easy as possible.

In some embodiments, the formulation may be a preparation form for a plant protection agent and/or pest control agent, consisting of one or more active ingredients and formulation auxiliaries, in which the biological effect of the active ingredient in the target organism is to be optimized and/or the finished product is to be brought into a form that is usable in technical equipment.

There are various ways of achieving the improved properties of a formulation (compared to the pure substance) for a defined application. Reference is made here to the extensive literature on formulation technology (see e.g.: M. J. Habib: Pharmaceutical Solid Dispersion Technology, Technomic Publishing Co., Inc., 2001, ISBN: 1-56676-813-6; T. Tadros: Formulation of Disperse Systems, Wiley 2014, ISBN: 978-3-5276-7830-3; T. F. Tadros: Formulation Science and Technology, De Gruyter 2018, ISBN: 978-3-1105-8759-3; T. G. Volova et al.: New Generation Formulations of Agrochemicals, Apple Academic Press, ISBN: 978-1-77188-749-6).

In some embodiments, the formulation may be a so-called “amorphous solid dispersion” (abbr.: ASD) in which a substance may be embedded in a water-soluble polymer and may be at least partially present there in amorphous form (formulation type: ASD). Amorphous solid dispersions are described, for example, in: N. Shah et al.: Amorphous Solid Dispersions, Springer 2014, ISBN: 978-1-4939-1597-2; P. J. Ghule: Amorphous solid dispersion: a promising technique for improving oral bioavailability of poorly water-soluble drugs, S. Afr. Pharm. J. 50, 2018, Vol. 85 No. 1; M. Müller et al.: Dissolution Behavior of Regorafenib Amorphous Solid Dispersion Under Biorelevant Conditions, 3rd European Conference on Pharmaceutics, 25-26 Mar. 2019, Bologna, Italy; G. Van den Mooter: The use of amorphous solid dispersions: A formulation strategy to overcome poor solubility and dissolution rate, Drug Discovery Today: Technologies. 2012, 9(2): e79-e85.

In some embodiments, the formulation may be a self-microemulsifying drug delivery system (abbr.: SMEDDS; formulation type: SMEDDS). Details on SMEDDS can be found, for example, in: Y. Qiu et al.: Developing Solid Oral Dosage Forms, Elsevier 2009, ISBN: 978-0-444-53242-8; D. J. Hauss: Oral Lipid-Based Formulations, CRC Press 2007, ISBN: 978-1-4200-1726-7; R. K. Tekade: Drug Delivery Systems, Academic Press 2020, ISBN: 978-0-12-814487-9; X. Ma et al.: Characterization of amourphous solid dispersions: An update, Journal of Drug Delivery Science and Technology Volume 50, April 2019, pages 113-124.

In some embodiments, the formulation may be a nanodispersion (formulation type: nanodispersion). Nanodispersions are described, for example, in: T. F. Tadros: Nanodispersions, De Gruyter 2015, ISBN: 978-3-11-029033-2; P. Nikansah et al.: Development and evaluation of novel solid nanodispersion system for oral delivery of poorly water-soluble drugs, J. Control. Release. 2013, 169(1-2): 150-161.

In some embodiments, the present invention may determine one or more properties (formulation properties) of at least one formulation for a substance with the aid of a computer system.

A computer system may be an electronic data processing system that processes data by way of programmable computing rules. Such a system usually comprises a computer, a processor for performing logic operations, and peripherals.

In computer technology, “peripherals” denotes all devices that are connected to the computer and are used to control the computer and/or as input and output devices. Examples thereof are monitor (screen), printer, scanner, mouse, keyboard, drives, camera, microphone, speaker and the like. Internal ports and expansion cards are also regarded as peripherals in computer technology.

Modern computer systems are frequently divided into desktop PCs, portable PCs, laptops, notebooks, netbooks, tablet PCs, handhelds (e.g., smartphones), cloud computers and workstations; all these systems can in principle be utilized for execution of the invention.

In some embodiments, a computer system according to the invention may comprise an input unit via which data and control commands can enter the computer system. Optionally, the computer system may comprise a data memory for storing data, in which data on substances (substance properties) in particular can be stored. In some embodiments, the computer system may comprise a control and computation unit (usually one or more processors and main memory) for controlling the individual components of the computer system, for coordinating the data flows, and for performing calculations, and an output unit via which data and information can be output from the computer system.

Data and/or control commands are usually input into the computer system via a keyboard, mouse, microphone, touch-sensitive display and/or the like. Data and information are usually output from the computer system via a monitor (screen), printer, data storage device, network connection (to another computer system) and/or the like.

In some embodiments, a substance may be specified in a first step. The substance can be specified, for example, by its name (e.g. IUPAC name, common name), by chemical structure (e.g. valence bond formula, perspective bond formula, structural formula, SMILES notation, chemical markup language or the like) and/or by another unique identifier (e.g. an alphanumeric code, a numeric code, a binary code, a bar or matrix code or the like).

In some embodiments, specification may be made by entering the unique identifier for the substance into a computer program, with selection from a list by a user also being understood as input.

For this substance it may be necessary to determine at least one formulation property of at least one formulation.

In some embodiments, in a further step, properties of the specified substance may be entered by a user and/or determined by the computer system, for example using the unique identifier from one or more data stores. Such a data store can be a component of the computer system according to the invention; however, in some embodiments, the computer system according to the invention can access such a data store via a network connection, for example, and can determine properties of the substance from there.

Properties of the substance (substance properties) are qualitative and/or quantitative features of the substance which characterize the substance and by which the substance can be distinguished from other substances.

In some embodiments, substance properties can be measured (empirically obtained) and/or calculated properties.

In some embodiments, substance properties may be physical and/or chemical and/or pharmacological and/or biological properties of the substance.

In some embodiments, at least some of the substance properties may be derived/calculated from the chemical structure of the substance. Chemoinformatics (also known as cheminformatics or chemical informatics) deals with methods for determining substance properties from the chemical structure; details on determining substance properties from the chemical structure can be found in numerous publications on this subject (see e.g.: B. A. Bunin et al.: Chemoinformatics: Theory, Practice & Products, Springer 2007, ISBN: 978-1-4020-5000-8; R. Guha et al.: Computational Approaches in Cheminformatic and Bioinformatics, Wiley 2011, ISBN: 978-0-470-3841-1; M. Karelson: Molecular descriptors in QSAR/QSPR, Wiley-Interscience 2000, ISBN: 978-0-4713-5168-9).

In some embodiments, substance properties (molecular properties) that may be determined and/or entered are: molecular weight, number of hydrogen bond donors, number of hydrogen bond acceptors, number of rotatable bonds, topological polar surface area, charge of the molecule, pKa values, number of different chemical groups: alcohols, carboxylic acids, acidic NH groups, esters, amide bonds, bases, alkyl radicals; solubility in water at pH 1, pH 4.5 and/or pH 7, hygroscopicity at 25° C./75%, glass transition temperature, melting point, viscosity in solution at 2%, 10%, 20%.

In some embodiments, the substance properties may be combined in a feature vector.

A feature vector may combine the (preferably numerically) parametrizable properties (features) of an object (a substance in the present case) in a vectorial manner Various features characteristic of the object may form the various dimensions of said vector. The entirety of possible feature vectors is called the feature space. Many machine learning algorithms require a numerical representation of objects since such representations facilitate or actually enable the processing of the data and statistical analysis. The generation of the feature vector thus serves to bring the substance properties determined and/or received into a form that enables computer-assisted processing.

Examples of generating feature vectors can be found in the prior art (see e.g. J. Frochte: Machine Learning, 2nd Edition, Hanser-Verlag 2019, ISBN: 978-3-446-45996-0).

In some embodiments, further values of parameters can be included in the feature vector, for example of parameters which characterize the formulation and/or formulating agent and/or any excipients and/or the medium into which the formulation is introduced in order to release the substance. In some embodiments, only substance properties and, for example, no properties relating to the formulation are included in the feature vector.

In some embodiments, the feature vector may then be fed into a prediction model. The prediction model may be configured to process the feature vector as input, to calculate one or more properties of at least one formulation of the substance (formulation property(ies)) using the feature vector, and to provide these as output variable(s) (output).

In some embodiments, the output of the one or more formulation property(ies) may, for example, be in alphanumeric form and/or graphical form on a monitor and/or on a printer, and/or the one or more calculated formulation property(ies) may be stored in a data memory.

In some embodiments, the prediction model may be a model that has been trained to calculate formulation properties based on substance properties using supervised learning methods on a training and validation data set. In some embodiments, the prediction model may be trained to correlate substance properties with formulation properties. Such a prediction model can then predict at least one formulation property of at least one formulation solely based on substance properties of a substance that is part of the formulation. In some embodiments, no data on the substance formulation are required for the prediction. In some embodiments, no experiments need to be carried out for the prediction. Experiments may, of course, be necessary for the generation (especially for training and validation) of such a prediction model. Reference formulations of reference substances must be generated, at least one formulation property of each reference formulation must be determined, and a model must be created that maps (measured and/or calculated) substance properties to the at least one formulation property of the reference formulations. The better the mapping, the higher the prediction accuracy. For the prediction of the at least one formulation property of a novel substance, however, once the prediction model has been trained, no experiments with the substance are required.

In some embodiments, two or more prediction models may be generated and may be available. A user can then, for example, specify the prediction model that they want to use for a prediction by means of an input. In some embodiments, the prediction model may be automatically determined and selected by the computer system by specifying a substance and/or a formulating agent and/or a formulation type and/or using another specified parameter.

In some embodiments, one prediction model may be generated for a specific formulation type. In some embodiments, the prediction model may be a prediction model for amorphous disperse dispersions (as a specific formulation type). some embodiments, the prediction model may be a prediction model for self-microemulsifying drug delivery systems (as a specific formulation type). In some embodiments, the prediction model may be a prediction model for nanodispersions (as a specific formulation type). In some embodiments, a prediction model may be generated for a formulation type with a specific type of carrier. For example, in some embodiments, a prediction model may be generated for amorphous disperse dispersions with a polymer from the family of cellulose ethers or polyethers or vinyl pyrrolidones or polyacrylic acid or for copolymers of methacrylic acid as carrier.

In some embodiments, the prediction model may be trained using a random forest method. A random forest method is a classification or regression method that consists of several uncorrelated decision trees. All decision trees have grown under a certain kind of randomization during the learning process. Details on generating a prediction model using a random forest method are described in the prior art (see e.g.: C. Sheppard: Tree-based Machine Learning Algorithms Decision Trees, Random Forests, and Boosting, CreateSpace Independent Publishing Platform 2017, ISBN: 978-1-9758-6097-4; T. W. Miller: Modeling Techniques in Predictive Analytics, Pearson Education, Inc., 2015, ISBN: 978-0-13-389206-2; P. Cichosz et al: Data Mining Algorithms, Wiley 2015, ISBN: 978-1-1183-3258-0).

In some embodiments, the prediction model may be, or may include, an artificial neural network. Such an artificial neural network may comprise at least three layers of processing elements: a first layer with input neurons (nodes), an N-th layer with at least one output neuron (nodes) and N-2 inner layers, where N is a natural number and greater than 2.

In some embodiments, the input neurons may serve to receive the values of the feature vector. In such a network, the at least one output neuron may be used to output at least one formulation property. The processing elements of the layers between the input neurons and the at least one output neuron may be connected to one another in a predetermined pattern with predetermined connection weights.

In some embodiments, the training of the neural network can, for example, be carried out by means of a backpropagation method. The aim here in respect of the network is maximum reliability of mapping of given input vectors onto given output vectors. The mapping quality is described by an error function. The goal may be to minimize the error function. In the case of the backpropagation method, an artificial neural network may be taught by the alteration of the connection weights.

In the trained state, the connection weights between the processing elements may contain information regarding the relationship between the substance properties (input) and the at least one formulation property (output), which can be used, for example, to predict the at least one formulation property for a novel substance.

A cross-validation method can be used in order to divide the data into training and validation data sets. The training data set may be used in the backpropagation training of network weights. The validation data set may be used to ascertain the prediction accuracy of the trained network.

Details on the creation and training of artificial neural networks are described, for example, in: G. Ciaburro et al.: Neural Networks with R, Packt Publishing 2017, ISBN: 978-1-78839-787-2; T. Rashid: Make Your Own Neural Network, O'Reilly 2016, ISBN: 978-1530826605.

In some embodiments, the prediction model may have been trained in a supervised learning process to learn, for a plurality of reference substances, a relationship between properties of the reference substances and properties of a large number of reference formulations, each reference formulation typically comprising a reference substance. In some embodiments, two or more of the reference formulations may include more than one reference substance.

The at least one predicted formulation property may be the concentration of the substance in a biologically relevant medium that occurs after a defined period of time when the formulation is introduced into the biologically relevant medium under defined conditions.

The defined conditions under which the formulation is introduced into the biologically relevant medium may be test conditions intended to mimic the conditions in the subsequent application. The specification of these defined conditions can include, for example, the following information: the biologically relevant medium used, the temperature of the medium, the pH of the medium, the stirring conditions, if any, and/or the like. The defined period of time can be, for example, one minute or a plurality of minutes or an hour or a plurality of hours. The concentrations of the substance in a biologically relevant medium may be predicted to occur after defined periods of time when the formulation is introduced into the biologically relevant medium under defined conditions. The prediction of the concentrations at two or more periods of time allows conclusions to be drawn about the dynamic dissolution behavior.

A biologically relevant (or biorelevant for short) medium may be a medium that occurs in a living organism or a medium that is artificially created in order to produce conditions that resemble (as closely as possible) the conditions of a medium that occurs in a living organism.

For example, since many medicaments are absorbed by the body via the gastrointestinal tract, a corresponding biorelevant media for the gastrointestinal tract aims to reproduce, for example, the gastrointestinal tract conditions in vitro, so that the behavior of medicaments and dosage forms in the gastrointestinal tract can be investigated in the laboratory. Typically, biorelevant media are used for in vitro solubility and dissolution studies; however, they can also be used for degradation studies or to determine the permeability properties of the medicament. Biorelevant media typically comprise solutions of chemical substances that naturally occur in the corresponding medium of the organism and are adjusted to pH values representative of the local area to be simulated.

Examples of biologically relevant media can be found in the prior art (see e.g.: K. Kleberg et al.: Characterising the behaviour of poorly water soluble drugs in the intestine: application of biorelevant media for solubility, dissolution and transport studies, JPP 2010, 62: 1656-1668; EP2645099A1; WO2008/040799A2).

In some embodiments, the following biologically relevant media are preferred: water, isotonic saline solution, FaSSGF, FaSSIF, FeSSIF, pharmaceutically typical buffer systems (e.g. phosphate buffer), and/or hydrochloric acid (preferably in a dilute, aqueous solution with a hydrochloric acid content corresponding to gastric juice).

FaSSGF stands for Fasted State Simulated Gastric Fluid and refers to biorelevant media for simulating physiological fluids under fasting conditions in the stomach of a human.

FaSSIF stands for Fasted State Simulated Intestinal Fluid and refers to biorelevant media for simulating physiological fluids under fasting conditions in the human intestine.

FeSSIF stands for Fed State Simulated Intestinal Fluid and refers to physiological conditions after food intake.

In the context of preclinical studies, biologically relevant media can also be used in the development of medicaments, which are intended to simulate conditions in a test animal, such as dog FaSSIF, dog FaSSGF, or rSIF (rat simulated intestinal fluid). In some embodiments, these are preferred biorelevant media in the context of the present invention.

In some embodiments, the prediction model may be configured to predict, for a substance, the properties of at least one formulation of that substance as an amorphous solid dispersion (ASD). In some embodiments, at least one formulation property of an amorphous solid dispersion based on one or more of the following carriers may be predicted: sugar (e.g. dextrose, sucrose, galactose, sorbitol, xylitol, mannitol, lactose), acid (citric acid, succinic acid, acetic acid), polymer (e.g. polyvinylpyrrolidone (PVP), polyethylene glycol (PEG), hydroxypropyl methylcellulose (HPMC), methylcellulose (MC), hydroxyethylcellulose, cyclodextrins, hydroxypropylcellulose, pectin, galactomannan, hydroxypropyl methylcellulose phthalate (HPMCP), Eudragit L100, Eudragit L100-55, Eudragit E100, Eudragit RL, Eudragit RS, Eudragit E PO, polyvinyl caprolactam-polyvinyl acetate-polyethylene glycol graft copolymer (e.g. Soluplus®) cellulose acetate phthalate, cellulose acetate butyrate, cellulose acetate, ethylcellulose, polyvinyl alcohol, copovidone PVP VA64, poly(styrenesulfonic acid), polyacrylic acid), surfactant (polyoxyethylene stearate, deoxycholic acid, polysorbate, macrogolglycerol, poloxamer (e.g. poloxamer 188), vitamin E, TPGS, sodium lauryl sulfate, macrogol(15) hydroxystearate, sorbitan ester), and/or others (e.g. pentaerythritol, pentaerythrityl tetraacetate, urea, urethane, hydroxyalkylxanthine).

In some embodiments, the invention may be implemented in such a way that a user can specify further parameters in addition to the unique identifier of the biologically active substance, which are then incorporated into the prediction model as further input variables. In some embodiments, for example, the at least one formulation may include a further biologically active substance and/or an auxiliary such as a surfactant in addition to the specified biologically active substance. In some embodiments, the at least one formulation property may be determined for a specific biorelevant medium and/or a specific pH of the medium and/or a specific active ingredient loading of a carrier and/or a specific maximum concentration of the active ingredient in the medium and/or the like. The prediction model can be configured to accept appropriate specifications as input values.

Depending on the configuration of the prediction model, for example, concentrations of an active ingredient in a biorelevant medium can thus be predicted at different time points and/or at different pH values and/or in different biorelevant media and/or at different active ingredient loadings.

In some embodiments, in each case at least one formulation property may be predicted for a plurality of formulations. The predicted formulation properties can be compared with one another. On the basis of the comparison, a sequence of the formulations can be formed according to their respective at least one formulation property. The sequence can reflect, for example, the increasing or decreasing solubility or the increasing or decreasing dissolution rate or the increasing or decreasing concentration in a biorelevant medium after a certain time or another property of the formulations. The sequence of the formulations can be output using an output unit (e.g. screen, printer). The at least one formulation property may be a measure in terms of quality and/or fitness for purpose. The formulations can be ranked according to their quality and/or suitability, e.g. the best and/or most suitable formulation first, the worst and/or most unsuitable formulation last and the remaining formulations ranked according to their quality and/or suitability. This allows a user to see directly which formulation(s) is (are) the most promising. The user can then concentrate on the most promising formulation(s), generate the respective formulation(s) and carry out investigations, for example, to verify the predicted formulation property(ies), for example. The present invention thus allows prioritization and a reduction in the number of experimental investigations.

In some embodiments, at least one formulation property may be predicted for at least one formulation and the at least one predicted formulation property is compared to at least one reference value. If the at least one formulation property deviates from the at least one reference value in a defined manner, a notification about the deviation can be output. The reference value can be, for example, an upper and/or a lower threshold value that the formulation may exhibit as a maximum or must at least fulfill in order to be usable for a purpose. With the comparison described, formulations can be checked directly with regard to their quality and/or applicability for a purpose. In some embodiments, only those formulations for which at least one formulation property is below the upper threshold value and/or above the lower threshold value are pursued.

In some embodiments, two or more formulation properties may be predicted for different formulations. For each formulation, at least one score value may be calculated from the formulation properties predicted for the formulation. The score value may be a measure of the quality and/or suitability of the respective formulation for a defined purpose. The formulations can be compared with one another and ranked based on their score values. The sequence can then be output. In some embodiments, the respective score values may be compared with reference values. If the score values deviate from the reference values in a defined manner, a notification about the deviation can be output.

The invention is more particularly elucidated below with reference to figures, without wishing to restrict the invention to the features and combinations of features that are shown in the figures.

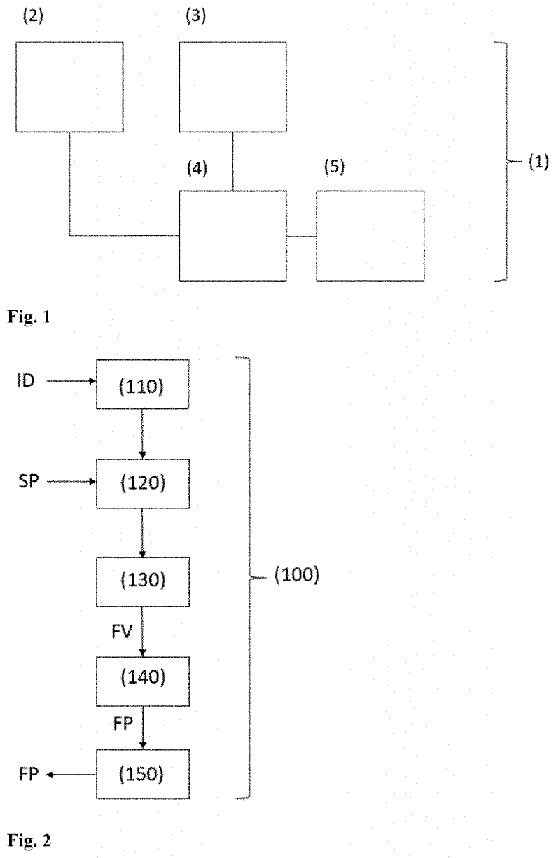

FIG. 1 shows an exemplary embodiment of the computer system according to some embodiments.

As shown, the computer system (1) may comprise an input unit (2), a data memory (3), a control and computation unit (4) and an output unit (5).

In some embodiments, a user may specify a substance via the input unit (2) for which at least one formulation property of at least one formulation that includes the substance as a constituent is to be predicted.

Optionally, further parameters can be entered by a user via the input unit (2), which specify the substance and/or the formulation and/or the use of the formulation in more detail. Examples of such further parameters are: substance properties, one or more auxiliaries in the formulation, one or more carriers in the formulation, one or more biologically relevant media in which the formulation is (to be) tested, one or more pH values of a medium in which the formulation is tested, loading of a carrier with the substance (active ingredient loading), one or more time points for which, for example, a concentration of the substance in a medium is to be predicted, and/or others.

Substance properties for a large number of substances may be stored in the data memory (3). In addition, data on formulations, formulation constituents, test conditions when investigating formulations and/or the like can be stored in the data memory (3). In some embodiments, there may be more than one data store (3), i.e., data used to predict at least one formulation property of at least one formulation can be distributed over two or more data memories. Each of these data memories may be part of the computer system according to the invention, or may be independent of the computer system according to the invention and (only) be connected to the computer system according to the invention via a connection (such as a network), so that the computer system according to the invention can read data from the data memory. In some embodiments, the computer system according to the invention may use one or more data memories in order to store results of the prediction in the data memory(ies).

The control and computation unit (4) may include one or more processors for performing calculations and logical operations (not explicitly shown). The control and computation unit (4) also includes a main memory in which a computer program (in particular the computer program according to the invention) can be loaded and executed (not explicitly shown).

The prediction model (or two or more prediction models) can also be loaded into the main memory. The prediction model may be part of the computer program according to the invention; it may include computer-readable instructions for performing calculations and logical operations.

The control and computation unit (4) may be configured (by means of the computer program according to the invention) to receive and/or determine substance properties for a specified substance and to generate a feature vector for the specified substance. The feature vector may serve as input to the prediction model. The prediction model may generate an output based on the feature vector. The output may be at least one formulation property of at least one formulation of the specified substance.

The at least one formulation property of the at least one formulation can be output by means of the output unit (5), e.g., displayed on a monitor, printed out on a printer, and/or stored in a data memory.

FIG. 2 shows an exemplary method which is carried out by the computer system according to some embodiments.

As shown, in some embodiments, the method (100) comprises the steps of:
(110) receiving a unique identifier (ID) of a biologically active substance,
(120) receiving and/or determining substance properties (SP) of the biologically active substance,
(130) generating a feature vector (FV) for the substance based on the substance properties (SP),
(140) determining at least one formulation property (FP) for at least one formulation of the substance using the feature vector (FV) by means of a prediction model, the prediction model having been trained in a supervised learning process to determine formulation properties for reference formulations using reference data from reference substances, and
(150) outputting the at least one formulation property (FP).

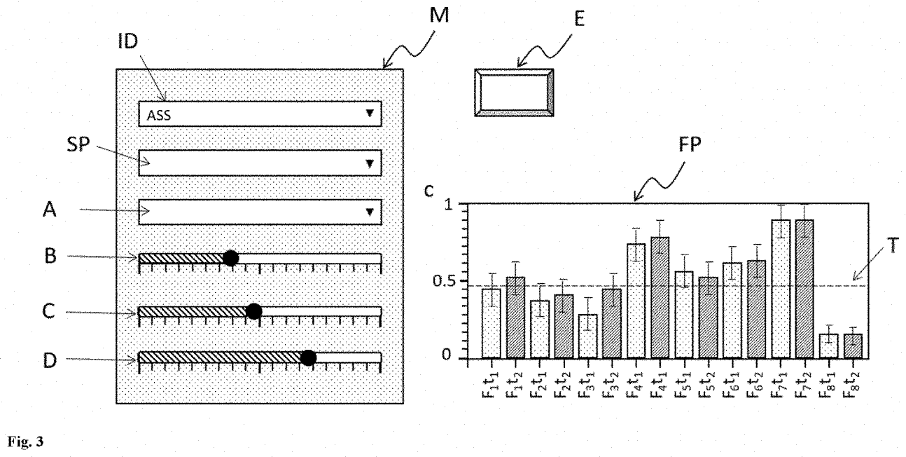

FIG. 3 shows an exemplary graphical user interface that can be provided by the computer system according to some embodiments.

The graphical user interface may include an input mask M via which a user can make inputs that are used for the prediction. In an input field, the substance, for which at least one formulation property (FP) of at least one formulation (Fₙ) may be predicted, may be specified by means of a unique identifier (ID). In the present case, the substance may be specified using the abbreviation of its name (ASS: acetylsalicylic acid). The name may be entered via a keyboard and/or selected from a list. Further substance properties (SP) may be entered in other fields.

Furthermore, inputs to the at least one formulation (Fn) and/or test conditions under which the at least one formulation can be tested in terms of its quality and/or suitability, may be entered via another input field (A) or via sliders (B, C, D). For example, the input field (A) could be used to specify a carrier and/or an auxiliary (e.g. a surfactant) or the like to be constituent(s) of the at least one formulation (Fn). In some embodiments, the (active ingredient) loading, i.e. the proportion by weight of the substance in the at least one formulation (Fn), the pH of the medium into which the at least one formulation (Fn) can be introduced for test purposes, and/or the like can be entered via the sliders (B, C, D).

After entering the various parameters, a user can start the calculation of the at least one formulation property (FP) of the at least one formulation by pressing a virtual switch button (E), for example. When the switch button (E) is activated, the computer system can, for example, read further substance properties using the unique identifier (ID), for example from a data memory, and use all substance properties and optionally further parameters (for example the parameters specified in the fields A, B, C and/or D) to generate a feature vector. The feature vector is then supplied as input to the one prediction model. The prediction model may be configured to determine at least one formulation property (FP) of at least one formulation (Fn) based on the feature vector. The at least one formulation property (FP) of the at least one formulation (Fn) can then be output. In the present case, for each of eight formulations (F1, F2, F3, F4, F5, F6, F7 and F8), the concentration c of the substance (ASS) that arises after two different time periods (t1, t2) when the respective formulation is introduced into a medium under defined conditions, is calculated and outputted. The respective concentrations may be shown graphically in the form of bar charts. On the abscissa, one bar for the time period t1 and one bar for the time period t2 are plotted for each formulation (Fn). The bars for the time periods t1 and t2 are hatched differently for better clarity. The concentration c is plotted on the ordinate in normalized form. The bar height thus shows the respective concentration c. Error bars indicate the level of accuracy of the prediction. A dashed line (T) marks a threshold value. In some embodiments, only those formulations where the concentration is above the threshold value for both time periods are further investigated.

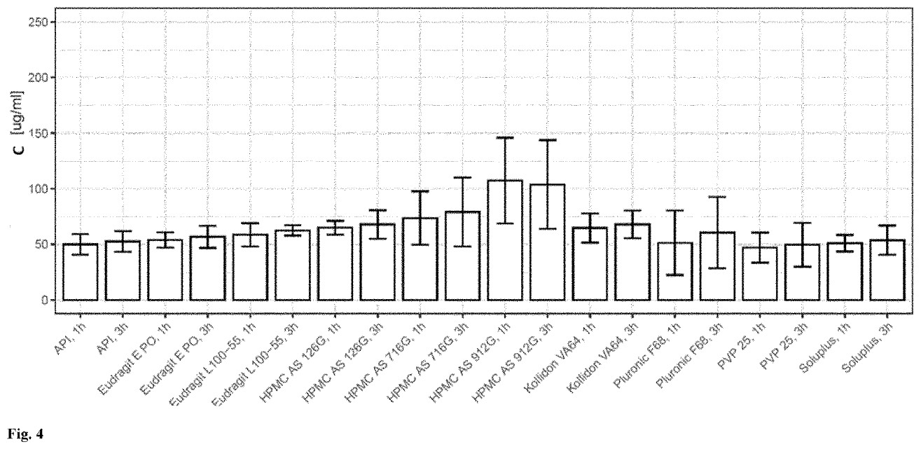

FIG. 4 shows an exemplary result of an exemplary prediction of formulation properties for a substance in the form of a graphical representation. The substance is the biologically active organic compound named nimodipine. The IUPAC name of nimodipine is 3-isopropyl 5-(2-methoxyethyl) 2,6-dimethyl-4-(3-nitrophenyl)-1,4-dihydropyridine-3,5-dicarboxylate; CAS number: 66085-59-4.

The graph shows the concentrations of nimodipine in a biologically relevant medium after two different time periods (after one hour: 1 h, and after three hours: 3h) for the pure substance nimodipine and for different formulations comprising nimodipine.

The pure substance is denoted by API.

The respective concentration C is plotted on the abscissa (y-axis) in units of micrograms per milliliter (μg/ml). The time periods (1 h, 3 h), the pure substance (API) and the respective formulations are plotted on the ordinate (x-axis).

The formulations are amorphous solid dispersions. The active ingredient loading, i.e. the proportion by weight of nimodipine in the formulations, was 20% in each case. The formulations are specified on the ordinate (x-axis) in terms of the carrier used (Eudragit E PO, Eudragit L100-55, HPMC AS 126G, etc.).

The predictions were made for FaSSIF as the biologically relevant medium. The conditions for which the predictions were made are the so-called standard conditions (temperature T=298.15K; pressure p=1.013 bar).

The predictions were obtained using a trained artificial neural network (see e.g.: https://arxiv.org/abs/1612.01474). For the training, the concentrations of various substances, which were introduced into various carriers as amorphous solid dispersions at a loading of 20% by weight, were determined experimentally after one hour and after three hours of stirring under standard conditions in FaSSIF. The network was trained to predict the determined concentrations from substance properties (in the present case molecular descriptors).

It can be seen in FIG. 4 that most formulations result in a higher concentration of nimodipine in FaSSIF. The highest concentrations are obtained for amorphous solid dispersions of nimodipine in HPMC AS 912 G. This formulation may be selected for verification of the predictions and for further experimental investigations. Formulations of nimodipine in, for example, PVP 25 show no increase in concentration and therefore do not need to be pursued further.

1. A computer system comprising:
a data and command input;
a data and information output; and
one or more processors configured to:
prompt the data and command input to receive a unique identifier of a substance,
determine substance properties based on the unique identifier,
generate a feature vector for the substance based on the substance properties,
using the feature vector, calculate at least one formulation property of at least one formulation using a prediction model, wherein the prediction model has been trained in a supervised learning process to calculate formulation properties for reference formulations using reference data from reference substances, and
prompt the data and information output to output the at least one formulation property.

2. The computer system of claim 1, wherein the at least one formulation is an amorphous solid dispersion of a biologically active substance in a carrier, or a nanodispersion of a biologically active substance, or a self-microemulsifying drug delivery system of a biologically active substance.

3. The computer system of claim 1, wherein the at least one formulation property is calculated exclusively from the substance properties, and wherein the substance properties are determined based on the chemical structure of the substance.

4. The computer system of claim 1, wherein the substance properties comprise one or more of: molecular weight, number of hydrogen bond donors, number of hydrogen bond acceptors, number of rotatable bonds, topological polar surface area, charge of the molecule, acid strength, number of defined chemical groups in the molecule, solubility in water, hygroscopicity, glass transition temperature, melting point and viscosity in solution at a defined concentration.

5. The computer system of claim 1, wherein the at least one formulation property is a concentration of the substance in a medium which occurs after a period of time when the formulation is introduced into the medium.

6. The computer system of claim 5, wherein the medium is a biologically relevant medium, and wherein the biologically relevant medium is preferably selected from the series: water, isotonic saline, FaSSGF, FaSSIF, FeSSIF, or hydrochloric acid.

7. The computer system of claim 1, wherein the at least one formulation property includes concentrations of the substance in a biologically relevant medium at or in one or more of: different time points, different pH values, different biorelevant media, and different active ingredient loadings.

8. The computer system of claim 1, wherein the prediction model is a regression model based on a random forest method.

9. The computer system of claim 1, wherein the prediction model is an artificial neural network or comprises an artificial neural network.

10. The computer system of claim 1, wherein the one or more processors are configured to compare the at least one calculated formulation property with a defined reference value and to output a result of the comparison.

11. The computer system of claim 1, wherein the one or more processors are configured to calculate two or more formulation properties for two or more substances, to determine a score value for each substance using the two or more calculated formulation properties and to output the score values.

12. The computer system of claim 1, wherein there are two or more prediction models for different types of formulation, and wherein the one or more processors are configured to prompt the data and command input, to receive information about a formulation and, based on the information received, to select a prediction model for calculating the at least one formulation property.

13. A method comprising:
receiving a unique identifier of a biologically active substance;
receiving and/or determining substance properties of the biologically active substance;
generating a feature vector for the substance based on the substance properties;
supplying the feature vector to a prediction model, wherein the prediction model has been trained in a supervised learning process to determine formulation properties for reference formulations using reference data from reference substances;
receiving at least one formulation property for at least one formulation comprising the biologically active substance as an output from the prediction model; and
outputting the at least one formulation property.

14. The method of claim 13, further comprising:
comparing the at least one formulation property or at least one score value calculated from the at least one formulation property with at least one reference value; and
in the case of a defined deviation from the at least one formulation property or the at least one score value from the at least one reference value: selecting the formulation for experimental verification of the at least one formulation property.

15. A non-transitory computer readable storage medium storing instructions configured to be executed by one or more processors of an electronic device, wherein, when executed by the one or more processors, the instructions cause the electronic device to:
receive a unique identifier of a substance;
receive and/or determine substance properties of the substance;
generate a feature vector for the substance based on the substance properties;
determine at least one formulation property for at least one formulation of the substance using the feature vector by means of a prediction model, wherein the prediction model has been trained in a supervised learning process to determine formulation properties for reference formulations using reference data from reference substances; and
output the at least one formulation property.
